(19) United States
(12) Patent Application Publication
(10) Pub. No.: US 2026/0123890 A1
(43) Pub. Date: May 7, 2026

(54) SLEEP MONITORING FRAMEWORK WITH ARTIFICIAL INTELLIGENCE

(71) Applicant: Ainnovation Labs, Inc, Brookline, MA (US)

(72) Inventors: Yun Fu, Brookline, MA (US); Zhi Xu, Cambridge, MA (US); Mingyuan Zhang, Malden, MA (US)

(73) Assignee: Ainnovation Labs, Inc, Brookline, MA (US)

(21) Appl. No.: 18/937,014

(22) Filed: Nov. 4, 2024

(51) Int. Cl. A61B 5/00; A61B 5/0205; A61B 5/024; A61B 5/08

(52) U.S. Cl. CPC A61B 5/7267; A61B 5/0205; A61B 5/4812; A61B 5/4815; A61B 5/4818; A61B 5/7203; A61B 5/7282; A61B 5/742; A61B 5/024; A61B 5/08; A61B 2505/07; A61B 2560/0247; A61B 2562/0204

(57) ABSTRACT

Embodiments of the present disclosure may include a sleep monitoring framework for detecting sleep stages and Sleep-Disordered Breathing (SDB), including an acoustic signal acquisition unit configured to collect raw acoustic signals from a subject, including respiration and heartbeat signals.

### BACKGROUND OF THE INVENTION

Embodiments of the present disclosure comprises a sleep monitoring framework for detecting sleep stages and Sleep-Disordered Breathing (SDB), including an acoustic signal acquisition unit configured to collect raw acoustic signals from a subject, including respiration and heartbeat signals.

### BRIEF SUMMARY

Embodiments of the present disclosure may include a sleep monitoring framework for detecting sleep stages and Sleep-Disordered Breathing (SDB), including an acoustic signal acquisition unit configured to collect raw acoustic signals from a subject, including respiration and heartbeat signals. Embodiments may also include a data preprocessing module coupled to the acoustic signal acquisition unit and adapted to normalize the raw acoustic signals into normalized signals by calculating maximum and minimum values of the raw acoustic signals and distributing the normalized signals to eliminate noise interference.

In some embodiments, normalization of the raw acoustic signals may be performed in two steps. In some embodiments, the two steps include a first step of mean removal and a second step of normalization by mean absolute error. Embodiments may also include a multi-scale deep learning network configured to process and analyze the normalized signals to extract features for detecting sleep stages and occurrences of apnea and hypopnea. In some embodiments, the sleep monitoring framework may be configured to operate without reliance on electrical signals such as Electrocardiogram (EKG) or Polysomnography (PSG).

In some embodiments, the sleep monitoring framework may be configured to be non-contact and suitable for home use and long-term monitoring. In some embodiments, the data preprocessing module may be configured to employ an adaptive algorithm to optimize signal normalization based on the environmental noise characteristics. In some embodiments, the multi-scale deep learning network includes an architecture configured to process both short-term and long-term acoustic signal features. In some embodiments, the multi-scale deep learning network may include modules, steps, or features such as Initial Convolution and Pooling, Multi-Scale Feature Extraction, Human Design Features. In some embodiments, the sleep monitoring framework for detecting sleep stages and Sleep-Disordered Breathing (SDB) may include a user interface configured to display sleep quality metrics based on the detected sleep stages and SDB occurrences.

Embodiments of the present disclosure may also include a method of monitoring sleep for detecting sleep stages and Sleep-Disordered Breathing (SDB), including acquiring raw acoustic signals from a subject. Embodiments may also include normalizing the raw acoustic signals into normalized signals to mitigate noise effects. Embodiments may also include extracting features from the normalized signals using a multi-scale deep learning network. Embodiments may also include classifying the extracted features to determine sleep stages and identify occurrences of apnea and hypopnea.

In some embodiments, the multi-scale deep learning network may be configured to use a set of human design features. In some embodiments, the set of human design features may include a set of features that may include Respiratory Rate interval, Heart Rate interval, Respiratory Rate peak time and Heart Rate peak time.

Embodiments may also include an adaptive algorithm may be used for normalizing the raw acoustic signals to mitigate noise effects. In some embodiments, the multi-scale deep learning network includes an architecture configured to process both short-term and long-term acoustic signal features. In some embodiments, the multi-scale deep learning network may include steps or features such as Initial Convolution and Pooling, Multi-Scale Feature Extraction, and Human Design Features. In some embodiments, the method of monitoring sleep for detecting sleep stages and Sleep-Disordered Breathing (SDB) may include displaying sleep quality metrics based on the detected sleep stages and SDB occurrences via a user interface.

Embodiments of the present disclosure may also include a sleep monitoring framework for detecting sleep stages and Sleep-Disordered Breathing (SDB), including an acoustic signal acquisition unit configured to collect raw acoustic signals from a subject, including respiration and heartbeat signals. Embodiments may also include a data preprocessing module coupled to the acoustic signal acquisition unit and adapted to normalize the raw acoustic signals into normalized signals by calculating maximum and minimum values of the raw acoustic signals and distributing the normalized signals to eliminate noise interference.

In some embodiments, normalization of the raw acoustic signals may be performed in two steps. In some embodiments, the two steps include a first step of mean removal and a second step of normalization by mean absolute error. Embodiments may also include a multi-scale deep learning network configured to process and analyze the normalized signals to extract features for detecting sleep stages and occurrences of apnea and hypopnea.

In some embodiments, the framework operates without reliance on electrical signals such as Electrocardiogram (EKG) or Polysomnography (PSG). In some embodiments, the multi-scale deep learning network may include modules, steps, or features such as Initial Convolution and Pooling, Multi-Scale Feature Extraction and Human Design Features.

In some embodiments, during the Initial Convolution and Pooling step, an input signal may be passed through a convolutional layer, followed by batch normalization, a ReLU activation, and max pooling. In some embodiments, the Initial Convolution and Pooling step may be configured to suppress the remaining noise. In some embodiments, during the Multi-Scale Feature Extraction step, the input signal may be processed with several functions and fed into a multi-scale feature extractor.

In some embodiments, the several functions may include function of MaxPool, Convolution, rectified linear unit (ReLu) and Batch Normalization. In some embodiments, the multi-scale feature extractor may be configured to output a set of features. In some embodiments, the set of features may be processed with another several steps. In some embodiments, the another several steps may include Feature Fusion, Dropout and Fully Connected (FC). In some embodiments, a set of outputs may be the results of the another several steps.

In some embodiments, the human design features may include a set of features that may include Respiratory Rate interval, Heart Rate interval, Respiratory Rate peak time and Heart Rate peak time. In some embodiments, the data preprocessing module employs an adaptive algorithm to optimize signal normalization based on the environmental noise characteristics.

In some embodiments, the multi-scale deep learning network includes an architecture configured to process both short-term and long-term acoustic signal features. In some embodiments, the sleep monitoring framework for detecting sleep stages and Sleep-Disordered Breathing (SDB) may include a user interface configured to display sleep quality metrics based on the detected sleep stages and SDB occurrences.

### BRIEF DESCRIPTION OF THE FIGURES

FIG. 1 is a block diagram illustrating a sleep monitoring framework, according to some embodiments of the present disclosure.

FIG. 2 is a flowchart illustrating a method of monitoring sleep, according to some embodiments of the present disclosure.

FIG. 3 is a block diagram illustrating a sleep monitoring framework, according to some embodiments of the present disclosure.

FIG. 4 is a block diagram further illustrating the sleep monitoring framework from FIG. 3, according to some embodiments of the present disclosure.

FIG. 5 is an example of a diagram of Preprocess the noisy signal by normalization

FIG. 6 is an example of a diagram of an overview of the disclosed sleep monitoring framework

FIG. 7 is an example of a diagram of Fusion of human design features and network extracted features

FIG. 8 is an example of a person sleeping on a bed with a nearby device coupled with the disclosed sleep monitoring framework

### DETAILED DESCRIPTION

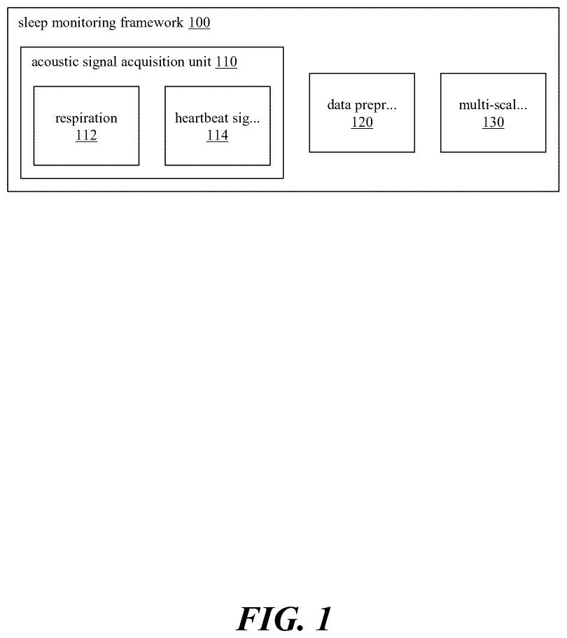

FIG. 1 is a block diagram that describes a sleep monitoring framework 100, according to some embodiments of the present disclosure. In some embodiments, the sleep monitoring framework 100 may include an acoustic signal acquisition unit 110 configured to collect raw acoustic signals from a subject, a data preprocessing module 120 coupled to the acoustic signal acquisition unit 110 and adapted to normalize the raw acoustic signals into normalized signals by calculating maximum and minimum values of the raw acoustic signals and distributing the normalized signals to eliminate noise interference, and a multi-scale deep learning network 130 configured to process and analyze the normalized signals to extract features for detecting sleep stages and occurrences of apnea and hypopnea.

In some embodiments, the acoustic signal acquisition unit 110 may include respiration 112 and heartbeat signals 114. Normalization of the raw acoustic signals may be performed in two steps. The two steps. A first step of mean removal and a second step of normalization by mean absolute error. The sleep monitoring framework 100 may be configured to operate without reliance on electrical signals such as Electrocardiogram (EKG) or Polysomnography (PSG).

In some embodiments, the sleep monitoring framework 100 may be configured to be non-contact and suitable for home use and long-term monitoring. In some embodiments, the data preprocessing module 120 may be configured to employ an adaptive algorithm to optimize signal normalization based on the environmental noise characteristics. In some embodiments, the multi-scale deep learning network 130 may include an architecture configured to process both short-term and long-term acoustic signal features. Steps or features such as Initial Convolution and Pooling, Multi-Scale Feature Extraction, Human Design Features. In some embodiments, the sleep monitoring framework 100 for detecting sleep stages and Sleep-Disordered Breathing.

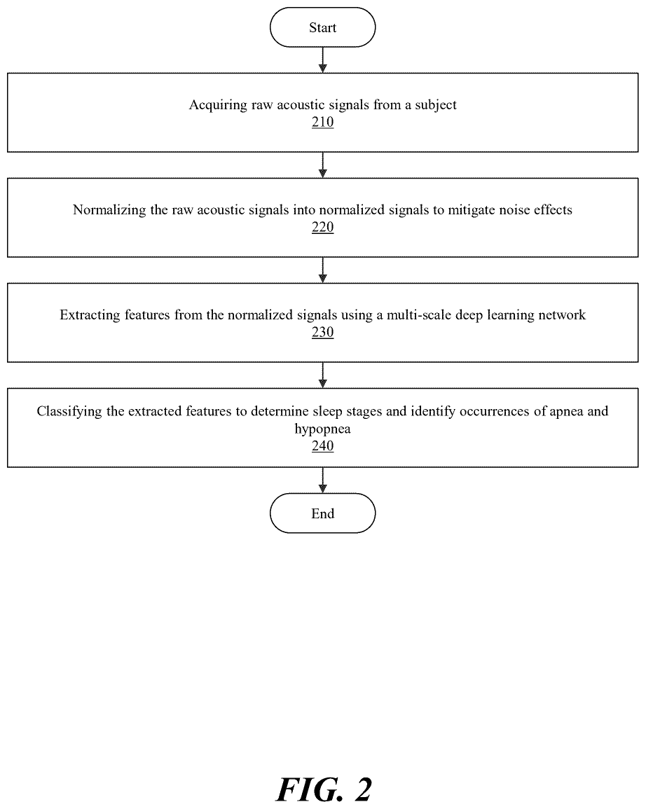

FIG. 2 is a flowchart that describes a method of monitoring sleep, according to some embodiments of the present disclosure. In some embodiments, at 210, the method may include acquiring raw acoustic signals from a subject. At 220, the method may include normalizing the raw acoustic signals into normalized signals to mitigate noise effects. At 230, the method may include extracting features from the normalized signals using a multi-scale deep learning network. At 240, the method may include classifying the extracted features to determine sleep stages and identify occurrences of apnea and hypopnea.

In some embodiments, the multi-scale deep learning network may be configured to use a set of human design features. The set of human design features may comprise a set of features that comprise Respiratory Rate interval, Heart Rate interval, Respiratory Rate peak time and Heart Rate peak time. In some embodiments, an adaptive algorithm may be used for normalizing the raw acoustic signals to mitigate noise effects. In some embodiments, the multi-scale deep learning network. The multi-scale deep learning network comprises steps or features such as Initial Convolution and Pooling, Multi-Scale Feature Extraction, and Human Design Features. In some embodiments, the method of monitoring sleep for detecting sleep stages and Sleep-Disordered Breathing

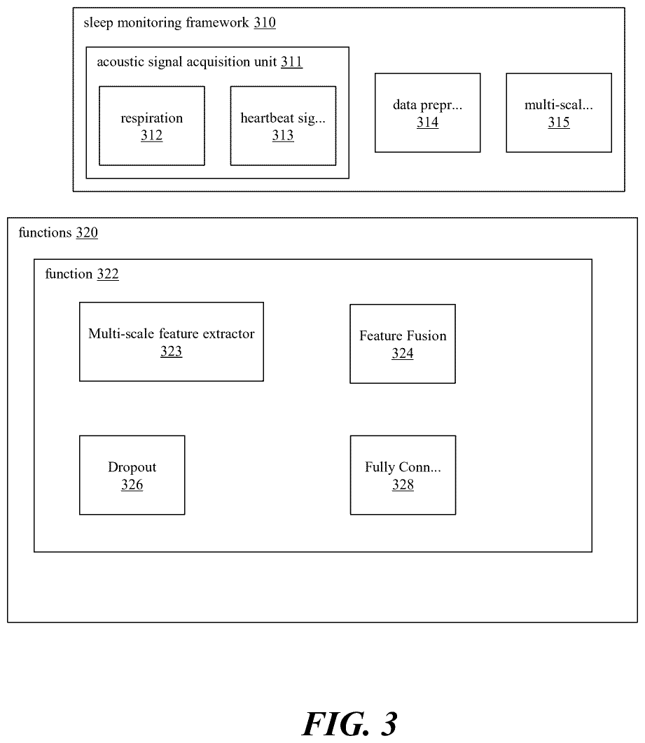

FIG. 3 is a block diagram that describes a sleep monitoring framework 310, according to some embodiments of the present disclosure. In some embodiments, the sleep monitoring framework 310 may include an acoustic signal acquisition unit 311 configured to collect raw acoustic signals from a subject, a data preprocessing module 314 coupled to the acoustic signal acquisition unit 311 and adapted to normalize the raw acoustic signals into normalized signals by calculating maximum and minimum values of the raw acoustic signals and distributing the normalized signals to eliminate noise interference, and a multi-scale deep learning network 315 configured to process and analyze the normalized signals to extract features for detecting sleep stages and occurrences of apnea and hypopnea.

In some embodiments, the acoustic signal acquisition unit 311 may include respiration 312 and heartbeat signals 313. Normalization of the raw acoustic signals may be performed in two steps. The two steps. A first step of mean removal and a second step of normalization by mean absolute error. The framework 310 may operate without reliance on electrical signals such as Electrocardiogram (EKG) or Polysomnography (PSG). Steps or features such as Initial Convolution and Pooling, Multi-Scale Feature Extraction and Human Design Features.

In some embodiments, following the Initial Convolution and Pooling step, an input signal may be passed through a convolutional layer, followed by batch normalization, a ReLU activation, and max pooling. The Initial Convolution and Pooling step may be configured to suppress the remaining noise. During the Multi-Scale Feature Extraction step, the input signal may be processed with several functions 320 and fed into a multi-scale feature extractor.

In some embodiments, the functions 320 may also include function 322 of MaxPool, Convolution, rectified linear unit (ReLu) and Batch Normalization. The function 322 may include a multi-scale feature extractor 323, Feature Fusion 324, Dropout 326, and Fully Connected 328 (FC). The multi-scale feature extractor may be configured to output a set of features. The set of features may be processed with another several steps. The another several steps. A set of outputs may be the results of the another several steps.

In some embodiments, the data preprocessing module 314 may employ an adaptive algorithm to optimize signal normalization based on the environmental noise characteristics. In some embodiments, the multi-scale deep learning network 315 may include an architecture configured to process both short-term and long-term acoustic signal features. In some embodiments, the sleep monitoring framework 310 for detecting sleep stages and Sleep-Disordered Breathing.

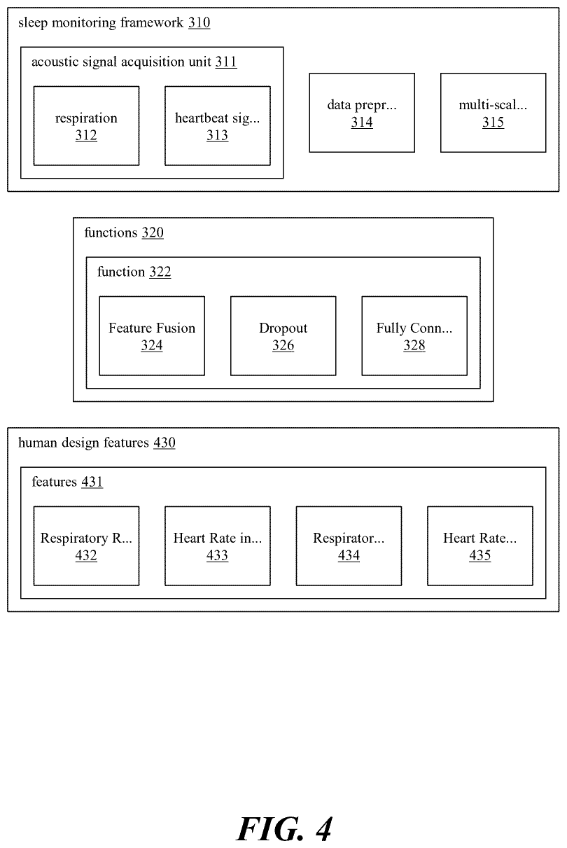

FIG. 4 is a block diagram that further describes the sleep monitoring framework 310 from FIG. 3, according to some embodiments of the present disclosure. In some embodiments, the human design features 430 may include a set of features 431 that. The set of features 431 may include Respiratory Rate interval 432, Heart Rate interval 433, Respiratory Rate peak time 434, and Heart Rate peak time 435.

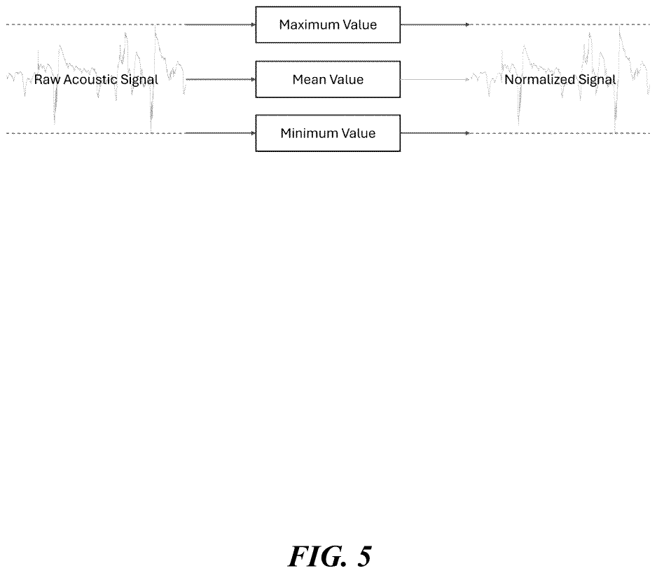

FIG. 5 is an example of a diagram of Preprocess the noisy signal by normalization.

The signal normalization is performed in two steps.
First step: Mean Removal:
Subtract the mean (μ) from the signal x_i:

$$signal\_normed = x \_i - µ$$

$$where µ = ( 1 / N ) * \sum ( x \_ i ) for i = 1 to N$$

Second Step: Normalization by Mean Absolute Error:
Normalize the mean-adjusted signal by dividing it by the mean absolute error (MAE):

$$signal\_normed = signal\_normed / MAE$$

$$where MAE = ( 1 / N ) * \sum \vert x\_i - µ \vert for i = 1 to N$$

The final normalization formula is:

$$signal\_normed = ( x\_i - µ ) / ( ( 1 / N ) * \sum \vert x\_i - µ \vert )$$

This process ensures that the signal is centered around zero and scaled to unit variance, facilitating further analysis.

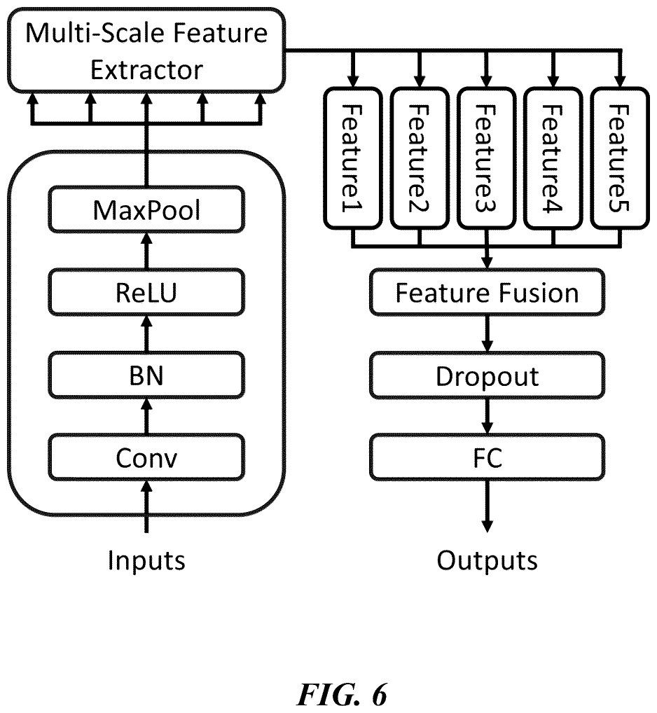

FIG. 6 is an example of a diagram of an overview of the disclosed sleep monitoring framework

In some embodiments, the framework comprises inputs going through steps of these following:
1) Initial Convolution and Pooling: The input signal x(t) is first passed through a convolutional layer, followed by batch normalization, a ReLU activation, and max pooling. This step reduces the temporal resolution suppresses the remaining noise.
2) Multi-Scale Feature Extraction: The model uses several parallel convolutional pathways with different kernel sizes. For each kernel size k, the signal x(t) is processed as follows:

$${Feature}_{x ( t )} = Max Pool ( {Conv}_{k} ( ReLU ( BN ( {Conv}_{k} ( x ( t ) ) ) ) )$$

Here, Convₖ denotes the convolutional layer with a kernel size of k, and Featureₓ₍ₜ₎ represents the feature map obtained from this pathway.
3) Human Design Features:
The model uses not only network-extracted features but also human design features. We use RR interval, HR interval, RR peak time and HR peak time as our human design features. After we get human design features, we use a feature fusion module to fuse them with the network-extracted features.

### Heart Rate (HR)

Heart rate is the number of times the heart beats per minute (bpm). During sleep, HR naturally decreases, particularly during deep NREM sleep, and becomes more variable in REM sleep. HR can increase significantly during apnea or hypopnea events as the body reacts to the oxygen deficit.

### Respiratory Rate (RR)

Respiratory rate is the number of breaths taken per minute (rpm). During sleep, RR decreases and becomes more regular in NREM sleep but can become irregular during REM sleep. In cases of apnea, RR pauses entirely, while in hypopnea, it becomes shallow and reduced.

### Relationship Between HR, RR, Sleep Stages, Apnea, and Hypopnea.

During NREM Sleep: Both HR and RR gradually slow down, reaching their lowest levels in deep sleep (N3). This stage is critical for bodily recovery and restoration.
During REM Sleep: HR and RR become more variable, reflecting the heightened brain activity associated with dreams.
During Apnea and Hypopnea: These breathing disruptions cause an immediate physiological response. HR increases to compensate for the reduced oxygen intake, and after the event, RR may temporarily spike to recover oxygen levels. These events can disrupt the sleep cycle, preventing individuals from reaching or maintaining deep sleep stages.
4) Feature Concatenation:
   The features extracted from each pathway are concatenated to form a comprehensive feature vector. This vector contains information from multiple temporal scales, enabling the model to consider both local and global patterns.
5) Classification:
   The concatenated features are passed through a dropout layer to prevent overfitting and then to a fully connected layer, which outputs the final class predictions:

$$Output = Softmax ( FC ( Dropout ( {Feature}_{concat} ( t ) ) ) )$$

Here, FC represents the fully connected layer, and Softmax converts the raw scores into probabilities for each class.
In essence, our model captures multi-scale features from the input signal by using multiple convolutional layers with different kernel sizes. These features are then combined to form a rich representation of the signal, which is used to make accurate predictions about sleep stages and SDB.

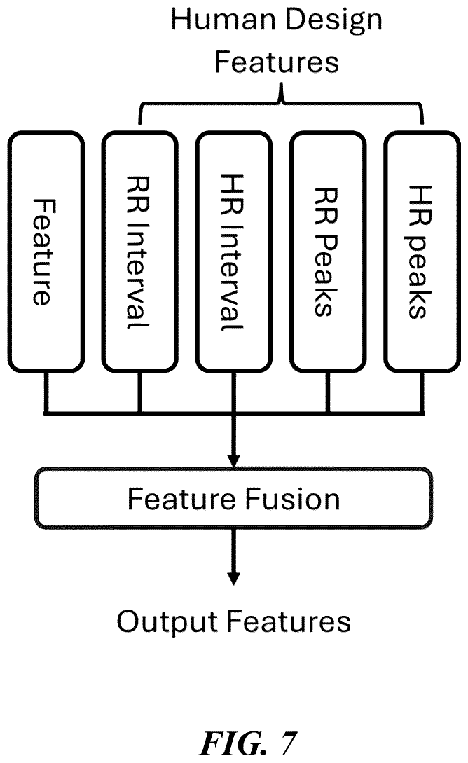

FIG. 7 is an example of a diagram of Fusion of human design features and network extracted features.

In some embodiments, Human Design Features are using following steps and features to realize its functions:
6) Human Design Features:
   The model uses not only network-extracted features but also human design features. We use RR interval, HR interval, RR peak time and HR peak time as our human design features. After we get human design features, we use a feature fusion module to fuse them with the network-extracted features.

### Heart Rate (HR)

Heart rate is the number of times the heart beats per minute (bpm). During sleep, HR naturally decreases, particularly during deep NREM sleep, and becomes more variable in REM sleep. HR can increase significantly during apnea or hypopnea events as the body reacts to the oxygen deficit.

### Respiratory Rate (RR)

Respiratory rate is the number of breaths taken per minute (rpm). During sleep, RR decreases and becomes more regular in NREM sleep but can become irregular during REM sleep. In cases of apnea, RR pauses entirely, while in hypopnea, it becomes shallow and reduced.

### Relationship Between HR, RR, Sleep Stages, Apnea, and Hypopnea.

During NREM Sleep: Both HR and RR gradually slow down, reaching their lowest levels in deep sleep (N3). This stage is critical for bodily recovery and restoration.
During REM Sleep: HR and RR become more variable, reflecting the heightened brain activity associated with dreams.
During Apnea and Hypopnea: These breathing disruptions cause an immediate physiological response. HR increases to compensate for the reduced oxygen intake, and after the event, RR may temporarily spike to recover oxygen levels. These events can disrupt the sleep cycle, preventing individuals from reaching or maintaining deep sleep stages.
7) Feature Concatenation:
   The features extracted from each pathway are concatenated to form a comprehensive feature vector. This vector contains information from multiple temporal scales, enabling the model to consider both local and global patterns.
8) Classification:
   The concatenated features are passed through a dropout layer to prevent overfitting and then to a fully connected layer, which outputs the final class predictions:

$$Output = Softmax ( FC ( Dropout ( {Feature}_{concat} ( t ) ) ) )$$

Here, FC represents the fully connected layer, and Softmax converts the raw scores into probabilities for each class.
In essence, our model captures multi-scale features from the input signal by using multiple convolutional layers with different kernel sizes. These features are then combined to form a rich representation of the signal, which is used to make accurate predictions about sleep stages and SDB.

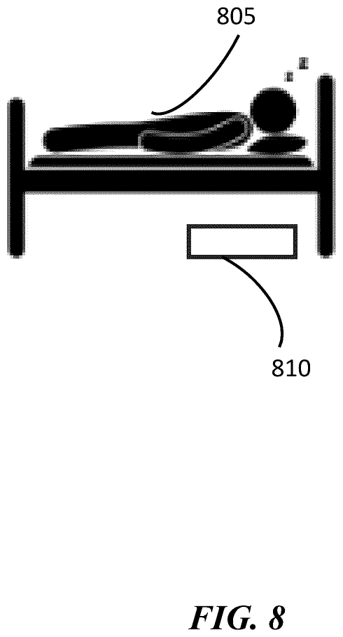

FIG. 8 is an example of a person sleep on a bed with a nearby device coupled with the disclosed sleep monitoring framework

In some embodiments, a person 805 sleeps on a bed with certain sleep stages. A device 810 with the disclosed sleep monitoring framework on its system to monitor the person 805's sleep conditions

In some embodiments, here the disclosure proposes an innovative algorithm for detecting sleep stages and Sleep-Disordered Breathing (SDB), including apnea and hypopnea, using heart rate and respiration rate data derived from acoustic signals during sleep. This approach diverges from mainstream methods that rely on electrical signals like Electrocardiogram (EKG) and Polysomnography (PSG). Our algorithm utilizes acoustic signals collected by devices such as those containing PVDF sensors. While acoustic signals present more noise challenges compared to electrical signals, they offer significant advantages in terms of ease of collection and resource efficiency. Our method employs an adaptive data preprocessing algorithm to normalize the data, mitigating the impact of unnecessary noise. Subsequently, we utilize a multi-scale deep learning network to process the normalized signals and generate results.

In addition, Acoustic Signals has these advantages and trade-offs: Non-contact, suitable for home environments or long-term monitoring, lower accuracy in detecting sleep stages and apnea, susceptible to environmental noise, indirectly reflects sleep stages, apnea, and hypopnea, ideal for non-invasive monitoring, easy to set up and use, very convenient, ideal for long-term, non-intrusive home monitoring, easily affected by environmental noise (e.g., fans, other people's voices), estimated indirectly through breathing sounds, lower accuracy.

In some embodiments, the approach leverages advanced acoustic signal processing techniques to overcome traditional limitations, such as noise interference and lower accuracy, providing a non-invasive, comfortable, and accurate solution for long-term sleep monitoring. Sleep is divided into two primary categories: NREM (Non-Rapid Eye Movement) sleep and REM (Rapid Eye Movement) sleep. NREM sleep can be further broken down into three stages (N1, N2, N3), while REM sleep represents the dream state. Each stage plays a vital role in the body's recovery and overall health. N1 (Light Sleep): This is the transition from wakefulness to sleep, with light, easily disrupted sleep. Breathing and heart rate begin to slow. N2 (Moderate Sleep): Breathing and heart rate continue to slow, and body temperature drops. This stage represents about 50% of total sleep time. N3 (Deep Sleep): Known as slow-wave sleep, it is the most restorative phase, where heart rate and breathing reach their lowest levels, and the body undergoes significant repair processes. REM Sleep: During REM sleep, heart rate and breathing become more variable, and brain activity increases. This phase is associated with vivid dreams and emotional memory consolidation.

In addition, related to Apnea/Hypopnea: Sleep apnea refers to the complete cessation of breathing for at least 10 seconds during sleep. There are two primary types:

Obstructive Sleep Apnea (OSA): This occurs due to the partial or complete blockage of the upper airway. It often leads to frequent awakening, gasping for air, and loud snoring. The body responds to the lack of oxygen by increasing heart rate (tachycardia). Central Sleep Apnea (CSA): This type occurs when the brain fails to send proper signals to the muscles controlling breathing, resulting in pauses in respiration. Hypopnea: Hypopnea is a partial blockage of the airway, characterized by reduced airflow (at least 30% reduction) and a corresponding drop in blood oxygen levels. While not as severe as apnea, hypopnea still disrupts sleep quality and can contribute to similar long-term health issues

Our raw acoustic data includes respiration and heartbeat signals from the human body, along with significant environmental white noise. To mitigate noise influence, we normalize the raw signals based on their maximum and minimum values, ensuring a uniform distribution. This preprocessing method eliminates most of the noise from the raw signal.

Our algorithm then separates different clean features from the processed signal using a multi-scale feature extractor to capture both short-term and long-term information. These features are used to detect sleep stages and the occurrence times of apnea and hypopnea during sleep.

1. A sleep monitoring framework for detecting sleep stages and Sleep-Disordered Breathing (SDB), comprising:
an acoustic signal acquisition unit configured to collect raw acoustic signals from a subject, including respiration and heartbeat signals;
a data preprocessing module coupled to the acoustic signal acquisition unit and adapted to normalize the raw acoustic signals by calculating maximum and minimum values of the signals and distributing the normalized signals to eliminate noise interference, wherein normalization of the raw acoustic signals is performed in two steps, wherein the two steps include a first step of mean removal and a second step of normalization by mean absolute error;
a multi-scale deep learning network configured to process and analyze the normalized signals to extract features for detecting sleep stages and occurrences of apnea and hypopnea, wherein the framework operates without reliance on electrical signals such as Electrocardiogram (EKG) or Polysomnography (PSG).

2. The sleep monitoring framework for detecting sleep stages and Sleep-Disordered Breathing (SDB) of claim 1, wherein the sleep monitoring framework is configured to be non-contact and suitable for home use and long-term monitoring.

3. The sleep monitoring framework for detecting sleep stages and Sleep-Disordered Breathing (SDB) of claim 1, wherein the data preprocessing module employs an adaptive algorithm to optimize signal normalization based on the environmental noise characteristics.

4. The sleep monitoring framework for detecting sleep stages and Sleep-Disordered Breathing (SDB) of claim 1, wherein the multi-scale deep learning network includes an architecture configured to process both short-term and long-term acoustic signal features, wherein the multi-scale deep learning network comprises steps or features such as Initial Convolution and Pooling, Multi-Scale Feature Extraction, Human Design Features.

5. The sleep monitoring framework for detecting sleep stages and Sleep-Disordered Breathing (SDB) of claim 1, further comprising a user interface configured to display sleep quality metrics based on the detected sleep stages and SDB occurrences.

6. A method of monitoring sleep for detecting sleep stages and Sleep-Disordered Breathing (SDB), comprising the steps of:
acquiring raw acoustic signals from a subject;
normalizing the raw acoustic signals to mitigate noise effects;
extracting features from the normalized signals using a multi-scale deep learning network; and
classifying the extracted features to determine sleep stages and identify occurrences of apnea and hypopnea.

7. The method of monitoring sleep for detecting sleep stages and Sleep-Disordered Breathing (SDB) of claim 6, wherein the multi-scale deep learning network is configured to use a set of human design features, wherein the set of human design features comprises a set of features that comprise Respiratory Rate interval, Heart Rate interval, Respiratory Rate peak time and Heart Rate peak time.

8. The method of monitoring sleep for detecting sleep stages and Sleep-Disordered Breathing (SDB) of claim 6, wherein an adaptive algorithm is used for normalizing the raw acoustic signals to mitigate noise effects.

9. The method of monitoring sleep for detecting sleep stages and Sleep-Disordered Breathing (SDB) of claim 6, wherein the multi-scale deep learning network includes an architecture configured to process both short-term and long-term acoustic signal features, wherein the multi-scale deep learning network comprises steps or features such as Initial Convolution and Pooling, Multi-Scale Feature Extraction, Human Design Features.

10. The method of monitoring sleep for detecting sleep stages and Sleep-Disordered Breathing (SDB) of claim 6, further comprising: displaying sleep quality metrics based on the detected sleep stages and SDB occurrences via a user interface.

11. A sleep monitoring framework for detecting sleep stages and Sleep-Disordered Breathing (SDB), comprising:
an acoustic signal acquisition unit configured to collect raw acoustic signals from a subject, including respiration and heartbeat signals;
a data preprocessing module coupled to the acoustic signal acquisition unit and adapted to normalize the raw acoustic signals by calculating maximum and minimum values of the signals and distributing the normalized signals to eliminate noise interference, wherein normalization of the raw acoustic signals is performed in two steps, wherein the two steps include a first step of mean removal and a second step of normalization by mean absolute error;
a multi-scale deep learning network configured to process and analyze the normalized signals to extract features for detecting sleep stages and occurrences of apnea and hypopnea, wherein the framework operates without reliance on electrical signals such as Electrocardiogram (EKG) or Polysomnography (PSG), wherein the multi-scale deep learning network comprises steps or features such as Initial Convolution and Pooling, Multi-Scale Feature Extraction and Human Design Features, wherein during the Initial Convolution and Pooling step, a input signal is passed through a convolutional layer, followed by batch normalization, a ReLU activation, and max pooling, wherein the Initial Convolution and Pooling step is configured to suppress the remaining noise, wherein during the Multi-Scale Feature Extraction step, the input signal is processed with several functions and fed into a multi-scale feature extractor, wherein the several functions comprises function of MaxPool, Convolution, rectified linear unit (ReLu) and Batch Normalization, wherein the multi-scale feature extractor is configured to output a set of features, wherein the set of features is processed with another several steps, wherein the another several steps comprise Feature Fusion, Dropout and Fully Connected (FC), wherein a set of outputs are the results of the another several steps.

12. The sleep monitoring framework for detecting sleep stages and Sleep-Disordered Breathing (SDB) of claim 11, wherein the human design features comprise a set of features that comprise Respiratory Rate interval, Heart Rate interval, Respiratory Rate peak time and Heart Rate peak time.

13. The sleep monitoring framework for detecting sleep stages and Sleep-Disordered Breathing (SDB) of claim 11, wherein the data preprocessing module employs an adaptive algorithm to optimize signal normalization based on the environmental noise characteristics.

14. The sleep monitoring framework for detecting sleep stages and Sleep-Disordered Breathing (SDB) of claim 11, wherein the multi-scale deep learning network includes an architecture configured to process both short-term and long-term acoustic signal features.

15. The sleep monitoring framework for detecting sleep stages and Sleep-Disordered Breathing (SDB) of claim 11, further comprising a user interface configured to display sleep quality metrics based on the detected sleep stages and SDB occurrences.
